(19) United States
(12) Patent Application Publication
(10) Pub. No.: US 2006/0210616 A1
(43) Pub. Date: Sep. 21, 2006

(54) Therapeutic patch for ophthalmologic and cosmetic use

(76) Inventor: Barry J. Linder, Danville, CA (US)

Correspondence Address: Barry J. Linder, 29 Bluehaven Court, Danville, CA, 94506, US

(21) Appl. No.: 11/083,881

(22) Filed: Mar. 17, 2005

(51) Int. Cl. A61K 9/70; A61F 13/00

(52) U.S. Cl. 424/449

(57) ABSTRACT

This invention is directed to a therapeutic patch for ophthalmologic and cosmetic use that provides therapy to the region where the patch is provided; the therapeutic patch provides a therapeutic delivery surface for placement against, for example, the eyelid and peri-orbital structures of a patient, and a non-contacting surface that can be used, for example, to generate heat and/or moisture for transmission to the surface to which therapy is being applied. The invention also includes a kit for at-home care of a region requiring therapy, in a manner that inhibits transmission of infectious agents.

### TECHNICAL FIELD

This invention relates a therapeutic patch for ophthalmologic and cosmetic use that provides therapy to the region where the patch is provided, specifically, the eye region; the therapeutic patch provides at least one therapeutic delivery layer for placement against, for example, the eye region including, e.g., the peri-orbital tissues of a patient, and a non-contacting layer that can be used, for example, to generate heat or moisture for transmission to the surface to which therapy is being applied. As contemplated by the invention, the patches can be pre-packaged in either sterile or non-sterile packing. The invention also includes a kit for at-home care of a region requiring therapy, in a manner that inhibits transmission of infectious agents.

### BACKGROUND

Eye patches are known in the art. For example, U.S. Pat. No. 4,682,371 discloses a protective eye patch. The '371 patch has several tabs for securing the patch to a patient's eye. U.S. Pat. No. 3,068,863 discloses a patch designed to keep the eye closed. U.S. Pat. No. 3,092,103 discloses a patch with a cushion material at the edge that allows the patient's eye to move underneath the eye patch. U.S. Pat. No. 3,908,645 for an ophthalmic therapeutic pressure bandage with a conformable, permeable carrier tape.

U.S. Pat. No. 6,409,746 discloses an eye pillow that releases steam from its surface applied to the eyes and the area around the eyes. The temperature is 50° C. or lower and has a total weight of 50 g or more.

Several conditions exist for which medical and cosmetic therapy is appropriate. For example, blepharitis, meibomitis, chalazia, and/or styes are common disorders of the eyelids that cause chronic inflammation in the peri-orbita, and are often associated with ocular tear film abnormalities resulting in dry eye symptoms. Symptoms of blepharitis include burning, itching, light sensitivity, and foreign body sensation. Signs include eyelash crusting, ocular discharge, eyelid scaling and swelling, and redness. For example, staphylcoccal blepharatis can have scaling and crusting along the eye lashes. There is no cure for blepharitis and long term treatment is required to keep it under control. Warm compresses, such as a warm washcloth, are used to heat the debris and crust on the lid for 5-10 minutes. After the lid has been warmed, a lid scrub is performed by using a suitable soap, such as Neutrogena or diluted Johnson's Baby Shampoo. Commercially available cleansing pads are available to assist in performing the lid scrub, for example OCuSOFT® Lid Scrubs or Novartis Ophthalmics Eye Scrub®. Following the eye scrub, antibiotics, such as polysporin, tobramycin, or erythromycin can be applied, to alleviate patient discomfort. Another condition for which therapy is appropriate is meibomitis, also known as the meibomian gland dysfunction. Meibomitis is a dysfunction of the meibomian gland that provides an oily layer as a critical component of the eye's natural tear film.

As reported by Olson et al, of the Schephens Eye Institute in Boston, warm moist compress therapy applied to the skin of the closed eyelids increases tear-film lipid layer thickness for subjects with meibodian gland dysfunction by more than 80% 5 minutes after initiating treatment and an additional 20% after 15 minutes of treatment. (See, PMID 12695712, Abstract: “Increase in tear film lipid layer thickness following treatment with warm compresses in patients with meibomian gland dysfunction.”).

What is needed is a device that conveniently delivers heat and moisture to at least one eye region, including the eyelids, and is held in place over one or both eyes with a biocompatible adhesive. Furthermore, this device, or an alternative embodiment of the device, can act as a platform for delivery of baby shampoo and any other therapeutic agent based on the type of treatment desired and the severity of the disease process, including for example, steroids, antibiotics and anti-wrinkle and anti-eye bag cosmetic formulations. Additionally, the device can provide a mechanism for easily determining when therapy is being delivered and when therapy is no longer being delivered.

### SUMMARY OF THE INVENTION

A variably configurable patch for delivering therapy to an eye region is provided that comprised: therapy delivery layer; an aperture for accessing the therapy layer; and an adhesive layer for adhering the patch to a surface. The patch can have a plurality of therapy delivery layers associated therewith. Additionally, the patch can comprise a therapeutic substance within the therapy delivery layer. Therapeutic substances can, for example, be selected from the group consisting of: baby shampoo, antibiotics, polysporin, tobramycin, steroids, prednisone, and anti-aging formulations. Additionally, a heat delivery layer can be provided. In the configuration with a heat delivery layer, it is contemplated that a non-permeable or permeable layer will separate the heat delivery layer from the therapy delivery layer. Additionally, a moisture delivery layer can be provided. As described above, a non-permeable or permeable layer can be provided to separate the moisture delivery layer from the heat delivery layer. Additionally, it is contemplated that layers, either permeable or non-permeable, can separate each of the layers, e.g., one or more therapeutic layers, one or more heat delivery layers, and one or more moisture delivery layers. One or more heat and/or moisture delivery layers might be desirable where heat and/or moisture is to be delivered on, for example, a time released scheduled. The patch of the invention can further comprise a non-irritating, hypoallergenic material which includes sterile or non-sterile water or saline or a therapeutic substance. Additionally, it is contemplated that the therapeutic substance might be activated by the application of heat from the heat delivery layer or moisture from the moisture delivery layer. It is also contemplated that the patch can include a mechanism for displaying, for example, heat delivery status, heat delivery temperature, absence of heat delivery, cooling delivery status, cooling delivery temperature, absence of cooling delivery, etc.

A pre-configured patch for delivering therapy to an eye is disclosed comprising: a layer selected from the group consisting of a heat delivery layer, a moisture delivery layer, and a therapy delivery layer; a skin contacting layer; an adhesive layer covering a portion of the skin contacting layer; and an exterior layer. As disclosed it is contemplated that the second layer is selected from the group consisting of a heat delivery layer, a moisture delivery layer, and a therapy delivery layer. Additionally, a plurality of therapy delivery layers can be provided. The patch can further comprise a therapeutic substance within the therapy delivery layer. Suitable therapeutic substances can include, for example, baby shampoo, antibiotics, polysporin, tobramycin, steroid creams or ointments, such as prednisone, and anti-aging formulations. This patch can, include a heat delivery layer and/or a moisture delivery and a non-permeable or permeable layer to separate the heat delivery layer from either the therapy delivery layer or the moisture delivery layer, or both. As contemplated, the therapeutic layer can be activated with the application of heat and/or moisture provided from either of the heat layer or the moisture layer of the patch. In any configuration, the therapy delivery layer, in contact with the skin, can contain any suitable therapeutic substance, including water or saline.

Kits are also provided for providing ophthalmologic or cosmetic treatment to an eye region housed in a container comprising: an eye patch; a therapeutic layer for an eye patch; an adhesive layer for an eye patch; a therapeutic substance provided within a container. Alternative kit compositions include a kit for providing ophthalmologic and/or cosmetic treatment to an eye region housed in a container comprising: a first eye patch for delivering heat and moisture to the eye region; and a second eye patch for delivering therapeutic substances to the eye region.

### BRIEF DESCRIPTION OF THE DRAWINGS

FIG. 1 is a top view of a patch according to the invention;

FIG. 2 is a lower view of a patch according to the invention;

FIGS. 3A-D are alternative cross-sectional views of the patch taken along the lines 3-3 in FIG. 1, wherein each figure illustrates an alternative embodiment;

FIG. 4 is a block diagram showing the steps of making a patch;

FIG. 5 is a top view of a patch, according to the invention, contained in packaging.

FIG. 6 is a top view of a patch, according to the invention, further including a display capable of indicating delivery of therapeutic temperature range.

### DETAILED DESCRIPTION

The following description is presented to enable any person skilled in the art to make and use the invention. Various modifications to the embodiments described will be readily apparent to those skilled in the art, and the generic principles defined herein can be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein. To the extent necessary to achieve a complete understanding of the invention disclosed, the specification and drawings of all issued patents, patent publications, and patent applications cited in this application are incorporated herein by reference.

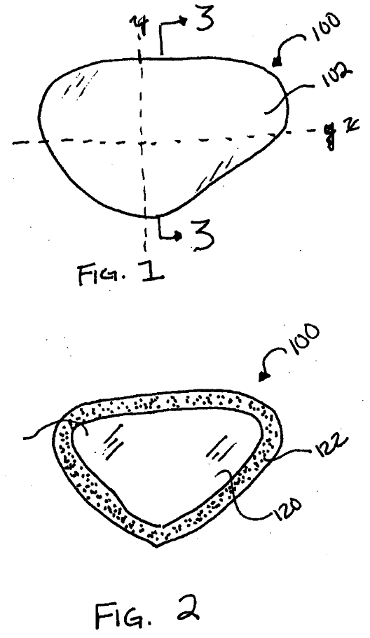

FIG. 1 illustrates a top view of a patch 100 according to the invention. As illustrated, the patch 100 can take on a tear-drop shape. Alternatively, the exterior perimeter of the patch 100 can take on other shapes, such as circular, substantially circular, oval, or substantially oval. The shape can be optimized for use on either the right or left eye, or the shape can be optimized for use with either eye. The overall width (measured along the x axis) and height (measured along the y axis) of the patch 100 will vary depending on the size of the patient for which the patch is intended. As shown here, the upper surface of the patch 102, which is not in contact with the patient, can be selected from the following materials: cotton, synthetic fibers, and/or suitable lint-free material. As will be appreciated by those of skill in the art, depending upon the contents of the various layers of the patch, it may be desirable for the upper surface 102 to be flexible while remaining fairly impermeable.

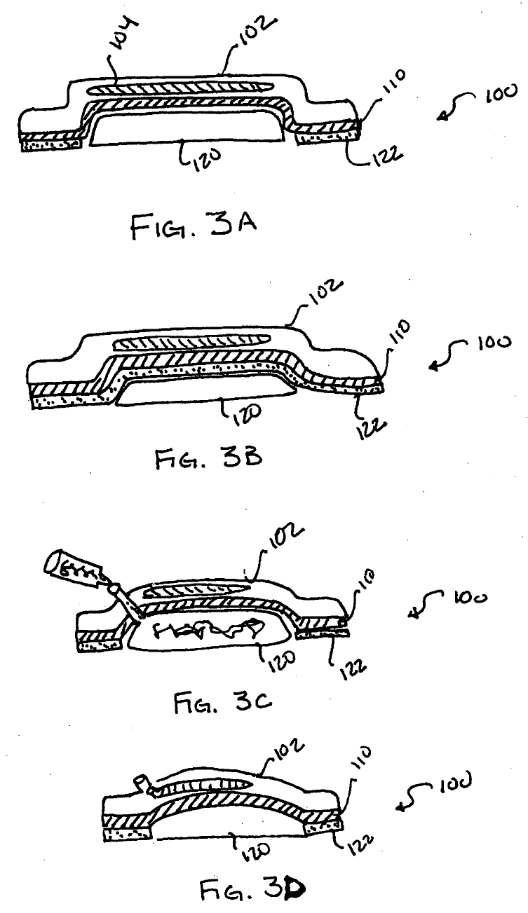

FIG. 2 illustrates a bottom view of a patch 100, showing the surface of the patch that is in contact with a patient's eyelid and periorbital skin, according to the invention. As illustrated, the patch 100 has a therapy delivery surface 120 and an adhesive edge 122 for adhering the patch to the skin surface of a patient. The adhesive can be formed of any suitable material that enables the patch 100 to adhere to the skin of the patient, including, for example, hypo-allergenic and latex free adhesive.

As will be appreciated by those of skill in the art, the patch can be assembled in a variety of configurations to achieve the intended result.

As shown in FIG. 3A, an upper surface 102 is provided which can provide a permeable or non-permeable seal for a delivery layer 104. The delivery layer 104 can be preformed to include, for example, water, polyethylene glycol, butylenes glycol, propylene glycol or iron powder. Alternatively, the delivery layer 104 can be a moisture delivery layer or a heated moisture delivery layer. In that event, the delivery layer 104, may be formed from two layers, one for generating heat and one for delivering moisture. As will be appreciated by those of skill in the art, delivery layer 104 can, in turn be formed of a plurality of components or layers enabling delivery of more than one therapy to be achieved. The mechanism of heat generation in layer 104 can be accomplished through several approaches. The heat can be generated as a byproduct of an exothermic chemical reaction. Reactants can include various combinations of Butylene Glycol, Sodium Silicoaluminate, Kaolin, PEG 8, Methyl Gluceth 20, Hydroxypropylcellulose, Talc, Acrylates Copolymer, Polyethylene, Methylcellulose, Ethylcellulose, BHT, Tetrasodium EDTA, and Ultramarines. Reactants such as these could be activated by exposure to water. An alternative approach could use iron filings, and could be activated by exposure to the oxygen in air. Another approach could be an external pouch that is activated via a microwave oven and inserted into layer 104. This pouch could be single or configured for multiple use.

Depending upon the contents of the delivery layer 104, a permeable or non-permeable layer 110 can be optionally provided. Where the delivery layer 104 is a heat layer, the optional layer 110 would most likely be non-permeable to the materials provided to generate heat. In that instance, the optional layer 110 would be formed of, for example, a water resistant material such as cellophane. Where the delivery layer 104 is designed to deliver only moisture, the optional layer 110, if present, would most likely be permeable to allow the moisture to reach the eye. In that instance, the optional layer 110 would be formed of any suitable material including, for example, cotton.

Depending upon the intended therapeutic application of the patch 100, the therapy delivery layer 120 could comprise a variety of materials. For example, a layer of hypoallergenic and/or non-irritating material with sterile or non-sterile water or saline could be provided; a layer of non-irritating material impregnated with a therapeutic substance could be provided; a layer of non-irritating material impregnated with a therapeutic substance activated with the application of heat could be provided; or a layer of non-irritating material impregnated with a therapeutic substance activated with the application of moisture could be provided. The non-irritating material could be any suitable material that is sterile or non-sterile, such as gauze. The impregnating substance could be, for example, baby shampoo; antibiotic, e.g., polysporin, tobramycin, etc.; steroids, e.g. prednisone; anti-aging treatment formulations, e.g., Eyeliss by Sederma, used to prevent puffiness and reduce bags under the eye, Drmazyl by Sederma, used as an anti-aging wrinkle smoothing and cutaneous barrier repair, Sudden Change Eye Gel with Green Tea by Sudden Change, Sudden Change Under-Eye Firming Serum, by Sudden Change, Bio-Performance Super Eye Contour Cream, an anti-wrinkle and anti-dark circle formulation by Shiseido, to name a few.

As illustrated in FIGS. 3A-B the adhesive edge 122 can be configured such that it encircles a portion of the outer edge of the patch 100 (shown in FIG. 3A). Alternatively, the adhesive layer 122 can be configured such that it forms a complete layer between the optional layer 110 and the delivery layer 120 (as shown in FIG. 3B).

In an alternative embodiment, shown in FIG. 3C, a receiving mechanism 140 is provided for filling the delivery layer 120. Such a mechanism is, for example, an aperture capable of enabling materials to be integrated into the layer 120. Ideally, the aperture would be sealable such that once the desired material is delivered to the therapeutic delivery layer 120, the aperture can be sealed to prevent ingress or egress of materials from the delivery layer 120. In this scenario, the delivery layer 120 could initially be dry and the user would inject, or otherwise permeate, the delivery layer 120 with the substance desired to come into contact with the skin surface. Alternatively, the layer could contain a fluid, such as water, or a catalyst for reacting with the materials delivered. Suitable substances are, for example, those listed above.

In an alternative embodiment, shown in FIG. 3D, a receiving mechanism 142 is provided for filling the delivery layer 104. Similar to the receiving mechanism 140 described above, a suitable mechanism is, for example, an aperture capable of receiving materials. In this scenario, the delivery layer 104 could be dry and the user would inject, or otherwise permeate, the delivery layer 104 with a substance capable of delivery heat, moisture or both to the patch. Suitable substances are, for example, those listed above.

In the embodiments described above it is contemplated that the filled materials can be inserted in a manner whereby the materials do not come in contact with the patient's skin, e.g., some of the filled materials used for generating heat and/or filled material that are not biocompatible. Additionally, it is contemplated that the patch 100 can be configured such that steam and/or heat is delivered for a prescribed period of time followed by delivery of therapeutic material. In this instance a degradable layer could be provided such that delivery of steam or heat degrades the layer of time such that the degraded layer allows for the delivery of therapeutic material. In this instance, the delivery of the therapeutic layer could occur some time after the beginning of delivering steam and/or heat or some time after the conclusion of delivering steam and/or heat. Persons of skill in the art would be familiar with materials suitable for achieving this objective.

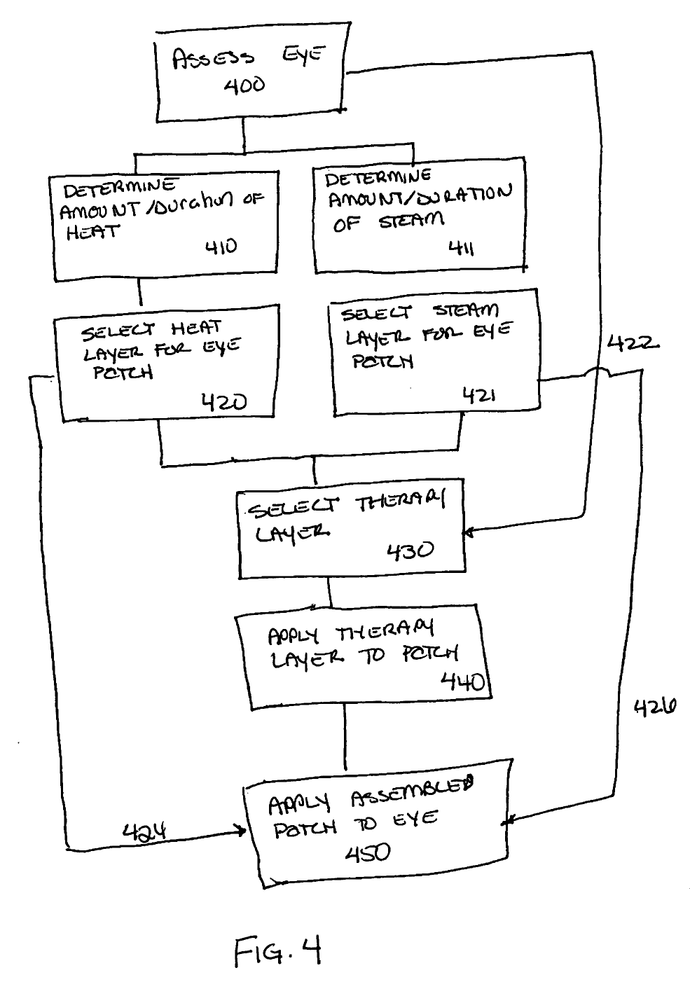

Turning now to FIG. 4, a flow chart is provided that illustrates the process for determining the configuration of a patch 100. The first step is to assess the eye and peri-orbital structures including the eye lids, eye lashes, and tear film 400. A variety of conditions suitable for use of an eye patch of this invention includes, for example, blepharitis, meibomitis, dry eye, rosacea, chalazia and conditions of aging of the peri-orbital structures such as eyelid wrinkles (including the condition commonly known as “crows' feet”) and eyelid “bags” or puffiness. In assessing the eye 400, a practitioner will determine the amount, if any, and duration of any heat to be applied 410 or steam to be applied 411. If heat is to be applied, then a suitable heat delivery mechanism or layer will be chosen 420. Alternatively, if steam is to be applied then a suitable steam delivery mechanism or layer will be chosen 421. As will be appreciated by those of skill in the art, theses steps can be skipped 422 without departing from the scope of the invention. Additionally, following the step of selecting a heat layer 420 or steam layer 421 the assembled eye patch can be applied to the eye 450 (steps 424 or 426)

In another process, after the eye is assessed 400, a therapy layer can be selected 430, and then applied to the eye patch 440. The assembled patch can then be applied to the eye 450.

When it is determined that both heat and therapy are required, the eye is assessed 400 as described above. The practitioner will then determine the amount and duration of heat to be applied 410 and will select a suitable heat delivery mechanism or layer 420. Thereafter, the practitioner can select a therapy layer 430 and apply the therapy layer to the eye patch 440. Once the eye patch has been configured, the assembled eye patch can then be applied to the eye 450. As will be appreciated by those of skill in the art, a practitioner can perform the process of selecting the therapy layer prior to selecting the heat layer without departing from the scope of the invention. Additionally, the process of selecting the layer and assembling the patch can be performed by the practitioner, e.g., physician or nurse treating a patient, or can be performed in a factory, or using a suitable manufacturing process, such that the practitioner selects a pre-made eye patch containing the desired heat layer and therapy layer and then applies the pre-assembled patch to the patient's eye. Alternatively, the process of selecting and assembling the patch can be performed by the patient. Additionally, whether selected by patient or practitioner, the patch can be self-applied by the patient.

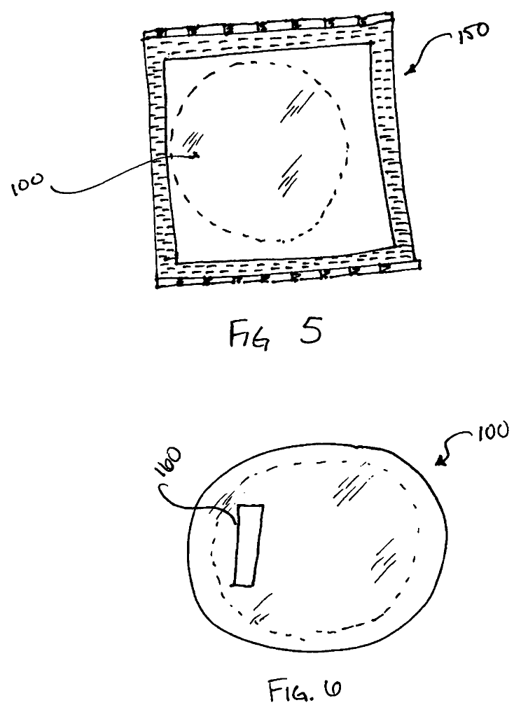

FIG. 5 illustrates a patch 100 which has been preconfigured and placed into a package 150 for delivery to a patient. The package can be sterile or non-sterile in configuration. The patch can be adhered using the adhesive edge 122 to the interior of the package 150 or can be placed on a removable insert within the package.

A variety of kits are contemplated with the invention. In a first kit, one or more patches 100 having the same therapeutic benefit can be provided, e.g. providing heat to warm the moisture and baby shampoo that are in the therapy delivery layer and intended to be in contact with the peri-orbital surfaces. Alternatively, a kit can be provided that provides one or more patches 100 having more than one benefit: e.g., a first type of patch with a first function, such as providing warmth and moisture to the eye, and a second type of patch with a second function, such as providing additional therapy to the surface. Yet another kit contemplated includes a plurality of blank patches 100 which can be loaded with a variety of materials, as described above, to achieve the intended therapeutic benefit.

Turning now to FIG. 6, an alternative embodiment of a therapeutic patch 100 of the invention is shown. In this embodiment, a therapy indicator 160 is provided. The therapy indicator can be, for example, a thermometer or decal with a temperature sensor that displays an indication of therapy status. Temperature sensing devices come in a variety of forms, and are known in the art. Temperature sensing devices include, for example, thermochromic liquid crystals (TLC), capable of changing colors in response to temperature. TLCs typically show color by selectively reflecting incident white light. The temperature sensitive mixtures in films then reflect colors as they turn from colorless (black against black background) to, for example, red at a given temperature or pass through other colors of the visible spectrum as the temperature increase. See, for example, U.S. Pat. No. 6,257,759. Where the patch 100 includes a therapy indicator 160, it is contemplated that the therapy indicator is adhered in a manner that protects the therapy indicator from ambient temperature and results in the indicator 160 providing an indication of the temperature of the therapy being delivered or the status of the therapy being delivered. The indicator 160 can, for example, provide a display that indicates the temperature, in either Celsius or Fahrenheit, of the therapy being delivered, or can indicate to the user the status of the patch in a “go-no go” fashion. If the go-no go indication is used, then the use of iconography, such as a “thumbs up” sign when the temperature is in the therapeutic range, and/or an “X” can be used to indicate that the therapy delivery cycle has completed (to accommodate non-English speaking patients), or the use of appropriate words, for example: Done, Fin, etc.

It is also contemplated that the therapy delivery indicator could function as a therapy delivery timer. In that instance, the indicator would indicate completion of therapy at the end of a prescribed period of time.

The foregoing description of embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention and the various embodiments and with various modifications that are suited to the particular use contemplated. It is intended that the scope of the invention be defined by the following claims and its equivalence.

What is claimed is:

1. A variably configurable patch for delivering therapy to an eye region comprising
a. a therapy delivery layer;
b. an aperture for accessing the therapy layer; and
c. an adhesive layer for adhering the patch to a surface.

2. The patch of claim 1 wherein a plurality of therapy delivery layers are provided.

3. The patch of claim 1 further comprising a therapeutic substance within the therapy delivery layer.

4. The patch of claim 3 wherein the therapeutic substance is selected from the group consisting of: baby shampoo, antibiotics that include polysporin and/or tobramycin, steroids that include prednisone, and anti-aging, anti-wrinkle and anti-eye bag formulations.

5. The patch of claim 1 further comprising a heat delivery layer.

6. The patch of claim 5 wherein a non-permeable layer separates the heat delivery layer from the therapy delivery layer.

7. The patch of claim 5 wherein a permeable layer separates the heat delivery layer from the therapy delivery layer.

8. The patch of claim 1 further comprising a moisture delivery layer.

9. The patch of claim 8 wherein a non-permeable layer separates the moisture delivery layer from the therapy delivery layer.

10. The patch of claim 8 wherein a permeable layer separates the moisture delivery layer from the therapy delivery layer.

11. The patch of claim 5 further comprising a moisture delivery layer.

12. The patch of claim 11 wherein a non-permeable layer separates the moisture delivery layer from the therapy delivery layer.

13. The patch of claim 11 wherein a permeable layer separates the moisture delivery layer from the therapy delivery layer.

14. The patch of claim 1 further comprising a layer of non-irritating material with sterile or non-sterile water.

15. The patch of claim 1 further comprising a layer of non-irritating material impregnated with a therapeutic substance.

16. The patch of claim 1 further comprising a layer of non-irritating material impregnated with a therapeutic substance activated with the application of heat.

17. The patch of claim 1 further comprising a layer of non-irritating material impregnated with a therapeutic substance activated with the application of moisture.

18. The patch of claim 1 further comprising a therapy status indicator.

19. The patch of claim 1 further comprising a therapy indicator.

20. The patch of claim 1 further comprising a therapy delivery timer.

21. A pre-configured patch for delivering therapy to an eye comprising:
a layer selected from the group consisting of a heat delivery layer, a moisture delivery layer, and a therapy delivery layer;
a skin contacting layer;
an adhesive layer covering a portion of the skin contacting layer; and
an exterior layer.

22. A pre-configured patch according to claim 21 wherein a second layer is selected from the group consisting of a heat delivery layer, a moisture delivery layer, and a therapy delivery layer.

23. The patch of claim 21 wherein a plurality of therapy delivery layers are provided.

24. The patch of claim 21 further comprising a therapeutic substance within the therapy delivery layer.

25. The patch of claim 24 wherein the therapeutic substance is selected from the group consisting of: baby shampoo, antibiotics that include polysporin, tobramycin, and steroids that include prednisone, and anti-aging, anti-wrinkle and anti-eye bag formulations.

26. The patch of claim 21 further comprising a heat delivery layer.

27. The patch of claim 26 wherein a non-permeable layer separates the heat delivery layer from the therapy delivery layer.

28. The patch of claim 26 wherein a permeable layer separates the heat delivery layer from the therapy delivery layer.

29. The patch of claim 26 further comprising a moisture delivery layer.

30. The patch of claim 29 wherein a non-permeable layer separates the moisture delivery layer from the therapy delivery layer.

31. The patch of claim 29 wherein a permeable layer separates the moisture delivery layer from the therapy delivery layer.

32. The patch of claim 26 further comprising a moisture delivery layer.

33. The patch of claim 32 wherein a non-permeable layer separates the moisture delivery layer from the therapy delivery layer.

34. The patch of claim 32 wherein a permeable layer separates the moisture delivery layer from the therapy delivery layer.

35. The patch of claim 21 further comprising a layer of non-irritating material with sterile or non-sterile water.

36. The patch of claim 21 further comprising a layer of non-irritating material impregnated with a therapeutic substance.

37. The patch of claim 21 further comprising a layer of non-irritating material impregnated with a therapeutic substance activated with the application of heat.

38. The patch of claim 21 further comprising a layer of non-irritating material impregnated with a therapeutic substance activated with the application of moisture.

39. The patch of claim 21 further comprising a therapy status indicator.

40. The patch of claim 39 wherein the therapy status indicator is configured to indicate the temperature of the therapy being delivered.

41. The patch of claim 39 wherein the therapy status indicator is configured to be protected from the ambient temperature.

42. The patch of claim 21 further comprising a therapy delivery timer.

43. A kit for providing ophthalmologic treatment to an eye region housed in a container comprising:
a. an eye patch;
b. a therapeutic layer for an eye patch;
c. an adhesive layer for an eye patch;
d. a therapeutic substance provided within a container.

44. The kit of claim 43 further comprising a therapy status indicator.

45. The patch of claim 44 wherein the therapy status indicator is configured to indicate the temperature of the therapy being delivered.

46. The patch of claim 44 wherein the therapy status indicator is configured to be protected from the ambient temperature.

47. The kit of claim 43 further comprising a therapy indicator.

48. The kit of claim 43 further comprising a therapy delivery timer.

49. A kit for providing ophthalmologic treatment to an eye region housed in a container comprising:
a. a first eye patch for delivering heat and moisture to the eye region; and
b. a second eye patch for delivering therapeutic substances to the eye region.

50. A kit for providing cosmetic treatment to an eye region housed in a container comprising:
a. a first eye patch for delivering heat and moisture to the eye region; and
b. a second eye patch for delivering cosmetically beneficial substances to the eye region.

51. A patch for delivering therapy to an eye region comprising
a. a therapy delivery layer;
b. an adhesive layer for adhering the patch to a surface; and
c. a therapy delivery indicator.

52. A pre-configured patch for delivering therapy to an eye comprising:
a layer selected from the group consisting of a heat delivery layer, a moisture delivery layer, and a therapy delivery layer;
a skin contacting layer;
an adhesive layer covering a portion of the skin contacting layer;
an exterior layer; and
a therapy delivery indicator.
